(19) United States
(12) Patent Application Publication
(10) Pub. No.: US 2026/0113573 A1
(43) Pub. Date: Apr. 23, 2026

(54) SYSTEM AND METHOD FOR COMPLEX AUDIO TONE DELIVERY THROUGH A PIEZOELECTRIC BUZZER

(71) Applicant: Medtronic, Inc., Minneapolis, MN (US)

(72) Inventors: Ilan D. Gordon, Crystal, MN (US); James E. Erler, Anoka, MN (US)

(21) Appl. No.: 19/353,149

(22) Filed: Oct. 8, 2025

Related U.S. Application Data
(60) Provisional application No. 63/708,472, filed on Oct. 17, 2024.

(51) Int. Cl. H04R 3/04; A61N 1/36; A61N 1/372; G10K 9/122

(52) U.S. Cl. CPC H04R 3/04; A61N 1/36125; A61N 1/37258; G10K 9/122

(57) ABSTRACT

A system, device, and method of using complex audio tones for device notifications. An audio device includes a non-volatile storage device storing binary pulse data, wherein the binary pulse data is generated by converting an audio file. The audio device also includes a direct memory access (DMA) engine that transfers the binary pulse data to at least one electronic component. The audio device includes at least one electronic component, which controls a duty cycle of an output signal to emit an audio output.

### CROSS-REFERENCE TO RELATED APPLICATIONS

This application claims the benefit of and priority to U.S. Provisional Patent Application No. 63/708,472 filed October 17, 2024, the entire disclosure of which is incorporated by reference herein.

### BACKGROUND

The present disclosure is generally directed to an audio device for delivery of auditory notifications and relates more particularly to an arrangement of one or more components of the audio device that allows for a more pleasant listening experience.

### BRIEF SUMMARY

Example aspects of the present disclosure include:

A method of generating audio, the method including converting an audio file into binary pulse data, wherein the binary pulse data is stored in a non-volatile storage; retrieving the binary pulse data from the non-volatile storage and populating a buffer in memory, transferring, via a direct memory access (DMA) engine, the binary pulse data stored in the buffer in memory to a timer controller of an audio device; and controlling a duty cycle of an output signal of the audio device based on the binary pulse data to emit an audio output.

An audio device including a non-volatile storage device that stores binary pulse data, wherein the binary pulse data is generated by converting an audio file; a central processing unit that retrieves the binary pulse data from the non-volatile storage device and populates a buffer in memory; a direct memory access (DMA) engine that transfers the binary pulse data stored in the buffer in memory to at least one electronic component; and the at least one electronic component that controls a duty cycle of an output signal of the audio device based on the binary pulse data to emit an audio output.

A wireless device including an audio device to: convert an audio file into binary pulse data, wherein the binary pulse data is stored in a non-volatile storage; retrieve, via a processor, the binary pulse data from the non-volatile storage and populate a buffer in memory transfer, via a direct memory access (DMA) engine, the binary pulse data from the buffer in memory to a timer controller of the audio device; and control a duty cycle of an output signal of the audio device based on the binary pulse data to emit an audio output.

Any of the aspects herein, wherein the audio device comprises a piezoelectric buzzer.

Any of the aspects herein, wherein the timer controller controls a voltage applied to the piezoelectric buzzer.

Any of the aspects herein, wherein the binary pulse data controls at least one of a frequency and a volume of the audio output.

Any of the aspects herein, wherein the audio device is included in an implantable device implanted in a patient.

Any of the aspects herein, wherein the audio device is included in a wireless device worn on the patient outside the body.

Any of the aspects herein, wherein the audio output comprises an auditory notification, and wherein each type of auditory notification corresponds to a different audio file.

Any of the aspects herein, further comprising: configuring, by a capture and compare register, the duty cycle of the output signal.

Any of the aspects herein, wherein pulse width modulation (PWM) is used to configure the duty cycle of the output signal.

Any of the aspects herein, wherein the audio output is outputted using a piezoelectric buzzer.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a block diagram of a system according to at least one embodiment of the present disclosure;

FIG. 1B is a block diagram of an audio device according to at least one embodiment of the present disclosure;

FIG. 2 is a flowchart illustrating a method of converting an audio file into signals to control an audio device according to at least one embodiment of the present disclose;

FIG. 3 illustrates various flowcharts according to at least one embodiment of the present disclosure;

FIG. 4 is a diagram illustrating an implantable device including the audio device according to at least one embodiment of the present disclosure;

FIG. 5 illustrates a wireless medical device according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), microprocessors used in embedded systems, graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

Patients may have a medical device implant. An implantable medical device is an instrument that is either wholly or partially introduced into the body. Most often, physicians implant these devices in surgery. Unlike surgical medical devices, implantable medical devices stay in the body after the procedure. An implantable medical device may need to provide notifications to the patient, and these notifications may include auditory notifications. Due to size and cost constraints, implantable medical devices may not have the capability for higher fidelity (speaker-based) playback.

Implantable medical devices may utilize piezoelectric buzzers for audio output. A piezoelectric buzzer is a type of electronic device that is used to produce a tone, an alarm, or a sound. A piezoelectric buzzer is lightweight with simple construction and is typically a low-cost product. Typical applications of a piezoelectric buzzer include alarms, warning devices, pest deterrents, electronic devices, and toys. Piezoelectric buzzers are known for shrill, sharp, and sometimes unpleasant auditory feedback due to their limited frequency response.

The present disclosure allows for a more complex tone delivery through a piezoelectric buzzer in order to provide a more pleasant audio experience for the patients. The present disclosure provides a better approximation of more complex wave tones by loading binary pulse data to a non-volatile storage mechanism at a high sample rate. An analog signal can be digitized without aliasing error if the sampling rate is greater than or equal to twice the highest frequency component in a given signal. In embodiments, the binary pulse data is sampled at a rate sufficient to approximate complex wave tones (e.g., at least 8 kHz). A Direct Memory Access (DMA) engine transfers the binary data in the non-volatile storage to a capture and compare register of a timer peripheral, wherein the capture and compare register is used to configure the duty cycle of the output signal. Controlling the duty cycle of the piezoelectric buzzer allows for control of the frequency and volume of the audio output.

The DMA engine allows hardware subsystems (e.g., the audio device) to access main memory without much monitoring from the central processing unit (CPU), which reduces the CPU's workload when a system needs to transfer large amounts of data from memory multiple times (e.g., transferring the binary pulse data each time an audio notification needs to be played). The present disclosure uses the DMA engine to transfer data from non-volatile memory to an audio device (e.g., a piezoelectric buzzer) for audio output. In other embodiments, the CPU retrieves a block of data from non-volatile memory and populates it in memory, and then points the DMA engine at that block in the memory

The present disclosure expands the possible sound palette, allowing for a wider range of possible tones and sequences, which may require large amounts of data, without added strain to the CPU. The present disclosure also enables ingestion from modern day audio file formats for rapid iteration during development. Audio files can be converted into binary data for load into non-volatile memory (e.g., an embedded MultiMediaCard (eMMC)), allowing users to stream selected audio files for delivery through the piezoelectric buzzer; and does not require a significant memory cost due to the rapid processing and retrieval of the binary data by the DMA engine, which offloads the processing from the main microprocessor, allowing the system to maintain responsivity to other user inputs or system inputs during the playback of audio tones.

This present disclosure allows for more rapid testing and development compared with previous design implementations. In the past, audio sequences were hardcoded values (frequency, volume, duration).

Embodiments of the present disclosure provide technical solutions to one or more of the problems of (1) improving sound quality from traditional playback methods used on previous instrumentation, due to a more precise control over the duty cycle values sent to a piezoelectric buzzer; (2) improving the audio output of an implantable device to provide a more pleasant listening experience for patients without the cost of a more complex audio chamber and speaker based solution, (3) retaining significant flexibility in the sound output, and (4) defining significantly more complex and specific waveforms, which allow users to perceive and associate a variety of connotations.

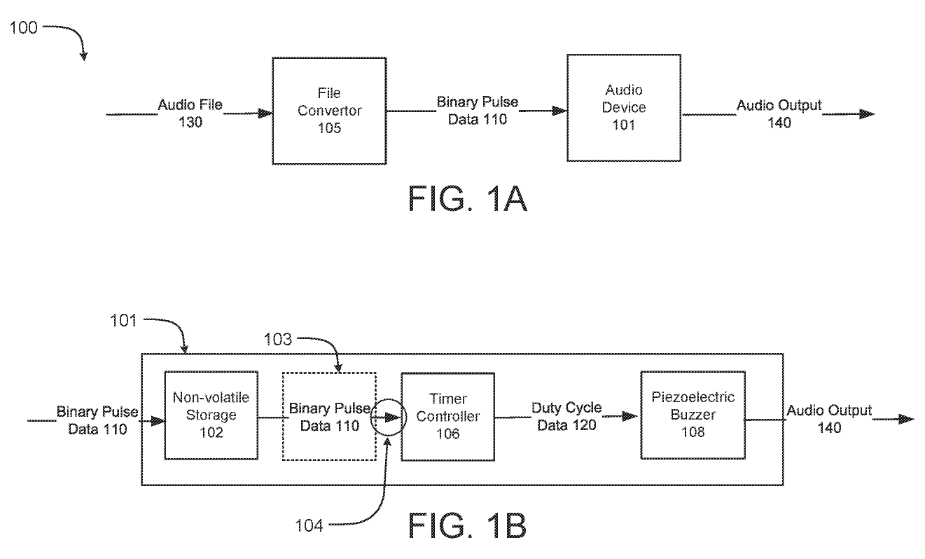

FIG. 1A depicts a block diagram of a system 100 for providing complex audio tone delivery of auditory notifications. System 100 includes a file converter 105 that converts an audio file 130 in binary pulse data 110. An audio device 101 uses the binary pulse data 110 to emit audio output 140. Although shown as separate from the audio device 101, the file converter 105 may be included in the audio device 101 or may be a separate device.

In embodiments, the audio output 140 comprises an auditory notification associated with an implantable medical device and different audio outputs 140 correspond to different notifications. For example, an implantable medical device may be a glucose monitor, and the glucose monitor may use different tones to alert the user of different conditions. Binary pulse data is another term that refers to data that has two possible values, often represented by the numbers 0 and 1, or “true” and “false.” Binary pulse data can be used to represent many things, such as: turning a voltage On/Off. In embodiments, the file converter 11 may use an algorithm to convert the audio file 130 in the binary pulse data 110. The binary pulse data 110 may be stored in the audio device 101. In embodiments, the binary pulse data 110 is stored in non-volatile storage 102 (e.g., Read Only Memory (ROM), eMMMC, etc.).

FIG. 1B illustrates the audio device 101 in more detail. As shown in FIG. 1B, the audio device 101 may include non-volatile storage 102, a buffer 103, a timer controller 106, and a piezoelectric buzzer 108. As described above, the non-volatile storage 102 may store the binary pulse data 110 that is generated by converting an audio file (e.g., the audio file 130). A processor (not shown) may retrieve the binary pulse data from the non-volatile storage 102 and populate a buffer 103 in memory. A DMA engine 104 transfers the binary pulse data 110 from the buffer 103 in memory to the timer controller 106. In embodiments, the timer controller 106 includes a capture and compare register. In capture mode, the capture and compare register stores a captured value from the specified signal; and in compare mode, the capture and compare register holds the value for comparison; while in Pulse Width Modulation (PWM) mode, this register is used to configure the duty cycle of the output signal. The duty cycle data 120 changes the timing of how long the output signal stays on and off.

The duration of the “on time” is called the pulse width, and changing the pulse width produces varying analog values. The percentage or ratio of how long the signal stays on compared to when it turns off is called the duty cycle. The timer controller 106 uses the duty cycle data 120 to control when the voltage is turned on/off to the piezoelectric buzzer 108, which creates the audio output 140. By controlling the pulse width of the voltage applied to the piezoelectric buzzer 108, the frequency and volume of the audio output 140 can be controlled to provide a more pleasant experience for the user/patient.

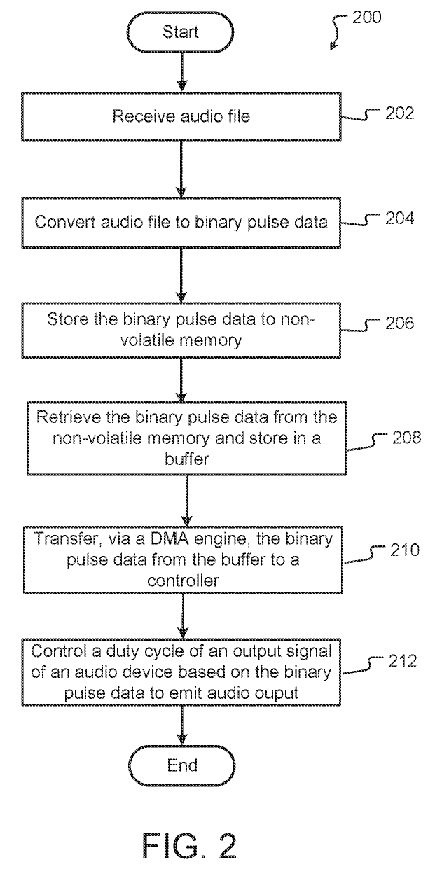

FIG. 2 depicts a method 200 providing complex audio tone delivery of auditory notifications. For example, the method 200 may be used to control the audio device 101. At step 202, an audio file is received. For example, the file converter 105 receives the audio file 130. In embodiments, the audio file 130 may comprise any sound file. The audio file may correspond to a popular song or be user generated. In embodiments, additional processing may be required to ensure that the tones in a song are still discernable after being delivered through the piezoelectric buzzer. At step 204, the audio file is converted into binary pulse data. For example, the file converter 105 converts the audio file 130 into binary pulse data 110. At step 206, the binary pulse data is stored in non-volatile memory (e.g., non-volatile storage 102). At step 208, the binary pulse data is retrieved from the non-volatile memory (e.g., non-volatile storage 102) and stored in a buffer in memory. At step 210, the binary pulse data is transferred from the buffer to a controller. For example, the binary pulse data 110 is transferred to the timer controller 106 using a DMA engine 104. In some embodiments, steps 208 and 210 may be combined into a single step, where the DMA engine 104 retrieves the binary pulse data from the non-volatile storage 102 and transfers the binary pulse data to the timer controller 106. At step 212, the controller controls the duty cycle of an output signal of an audio device based on the binary pulse data to emit audio output. For example, the DMA engine 104 transfers the binary pulse data 110 to a capture and compare register in the timer controller 106, the timer controller 106 controls the duty cycle of the piezoelectric buzzer 108 to emit audio output 140. For example, the timer controller 106 controls the timing of when voltage is applied to the piezoelectric buzzer 108. In embodiments, the audio output 140 comprises an auditory notification associated with an implantable medical device and different audio outputs 140 correspond to different notifications.

The present disclosure encompasses embodiments of the method 200 that comprise more or fewer steps than those described above, and/or one or more steps that are different than the steps described above.

As noted above, the present disclosure encompasses methods with fewer than all of the steps identified in FIG. 2 (and the corresponding description of the method 200), as well as methods that include additional steps beyond those identified in FIG. 2 (and the corresponding description of the method 200). The present disclosure also encompasses methods that comprise one or more steps from one method described herein, and one or more steps from another method described herein.

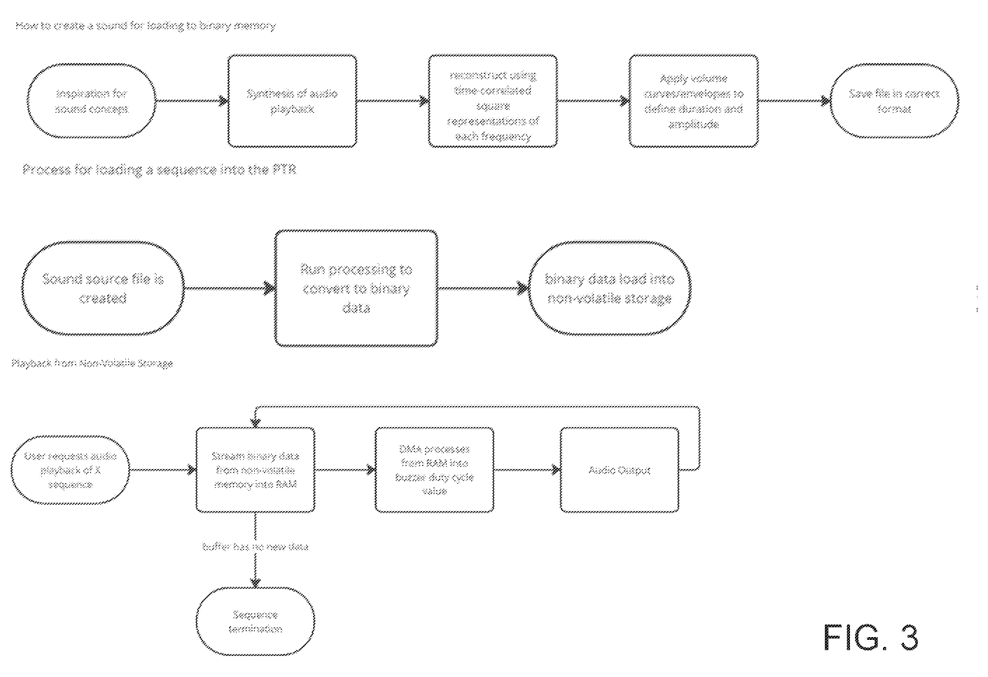

FIG. 3 illustrates various processes included in providing complex audio tone delivery of auditory notifications. The processes include how to create a sound that is saved to the non-volatile memory of an audio device. Another process is illustrated for playback of the sound from the non-volatile memory of the audio device.

The process of creating a sound starts by having an inspiration of a sound concept. Once the sound concept is determined, the sound concept can be synthesized or reconstructed using time-correlated square representations of each frequency. Then volume envelopes are applied to define the duration and amplitude. Then the file is saved in the correct format for use by the audio device. Through this more dynamic manipulation of the amplitude over time, playback output can take on a range of timbres.

The process of loading the sound into the audio device includes creating the sound source file. In embodiments, the sound source file may be an existing audio file. The sound source file is processed to convert the sound source file into binary data (e.g., binary pulse data). The binary data is saved to the audio device.

The process of playing the audio includes streaming the binary data from the non-volatile storage into RAM, processing, by a DMA engine the binary data into duty cycle values for the piezoelectric buzzer and outputting the audio, the process repeats until all the audio data is outputted.

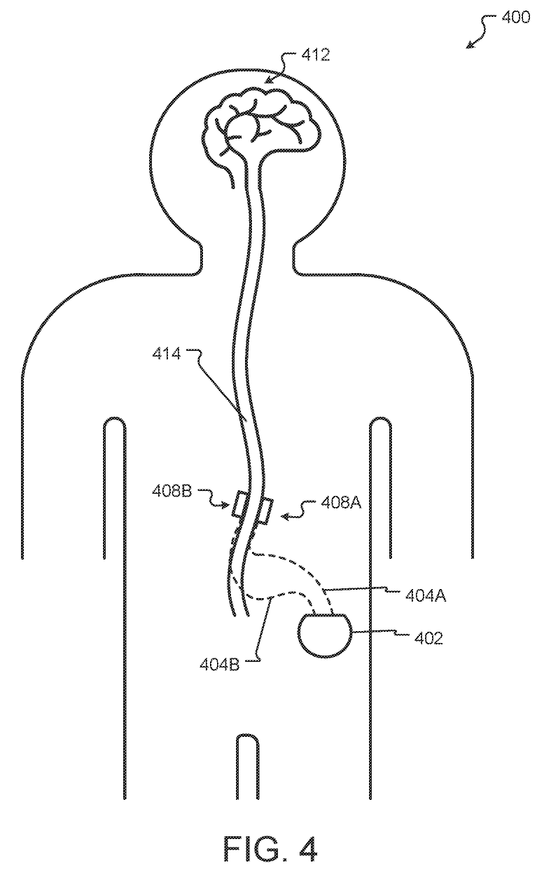

FIG. 4 illustrates a patient 400 that has an implantable medical device 402. The implantable medical device 402 may be used to provide electric signals via pads or electrodes 408A-B to a patient and/or carry out one or more other aspects of one or more of the methods disclosed herein. In embodiments, the audio device may be included in a medical device that is worn by the patient, rather than implanted. In one example, the implantable medical device 402 provides neuromodulation techniques (e.g., technologies that act directly upon nerves of a patient, such as the alteration, or “modulation,” of nerve activity by delivering electrical impulses directly to a target area) for assisting in treatments for different diseases, disorders, or ailments (e.g., chronic pain) of a patient. As discussed herein, neuromodulation techniques may be used to relieve chronic pain. Additionally or alternatively, neuromodulation techniques may be used to stimulate or prevent other neurological signals from traveling to or from the patient’s brain for the purposes of assisting with patient treatment.

The implantable medical device 402 may be used for, for example, a close-loop or open spinal cord stimulation, deep brain stimulation, pelvic health, etc. that is capable of providing a stimulation to a target anatomical element. In the illustrated embodiment, the target anatomical element is a spinal cord 414 of the patient 400, though in other embodiments the target anatomical element may be, for example, a brain 412 of the patient 400 and/or one or more nerve endings of the patient. In some examples, the implantable medical device 402 may be referred to as a close-loop stimulator, an open-loop stimulator, a pulse generator, an implantable neural stimulator, an internal neural stimulator, or the like, which may be implantable in some embodiments. More specifically, the implantable medical device 402 may be configured to generate a current or electrical signal that is delivered to the target anatomical element. In other embodiments, the implantable medical device 402 may be external to the patient.

Additionally, the patient 400 may include one or more leads 404A-B (e.g., electrical leads) that provide a connection between the implantable medical device 402 and the spinal cord or nerves of the patient 400 for enabling, for example, stimulation.

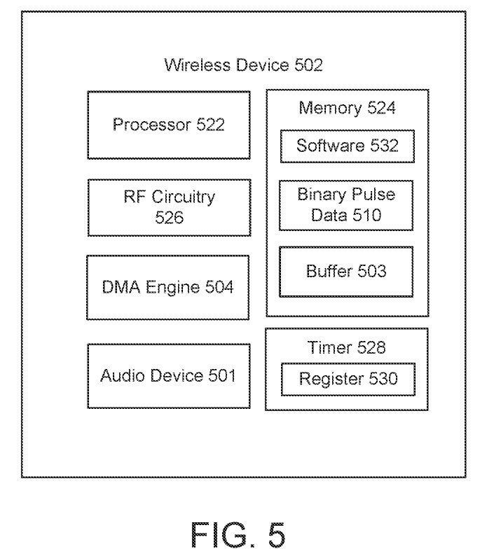

Turning now to FIG. 5, which illustrates an example wireless medical device 502. In embodiments, the wireless medical device 502 may be worn by a patient or implanted in the patient’s body. The medical device 502 includes a processor 522, a memory 524, a radio frequency (RF) circuitry 526, a timer 528, DMA engine 504, and an audio device 501.

Processor 522 comprises a microprocessor and other circuitry that retrieves and executes operating software 532 from the memory 524. Memory 524 comprises a non-transitory storage medium, such as a disk drive, flash drive, data storage circuitry, or some other memory apparatus and a buffer 503. The processor 522 may be mounted on a circuit board that may also hold memory 524. Operating software 532 comprises computer programs, firmware, or some other form of machine-readable processing instructions. Operating software 532 may include an operating system, utilities, drivers, network interfaces, applications, or some other type of software. When executed by the processor 522, operating software 532 directs the medical device 502 to operate. Radio frequency (RF) circuitry 526 typically includes an antenna, amplifier, filter, modulator, and signal processing circuitry for receiving and transferring wireless signals. Binary pulse data is stored in non-volatile memory 510 in the memory 524. The timer 528 uses the binary pulse data to control the duty cycle of the audio device 501 to emit audio output (e.g., audio output140). For example, the binary pulse data may be transferred to the register 530 (e.g., a capture and control register), that uses PWM to control the duty cycle of the audio device 501 (e.g., a piezoelectric buzzer).

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method of generating audio, the method comprising:
converting an audio file into binary pulse data, wherein the binary pulse data is stored in a non-volatile storage;
retrieving the binary pulse data from the non-volatile storage and populating a buffer in memory;
transferring, via a direct memory access (DMA) engine, the binary pulse data stored in the non-volatile storage to a timer controller of an audio device; and
controlling a duty cycle of an output signal of the audio device based on the binary pulse data to emit an audio output.

2. The method of claim 1, wherein the audio device comprises a piezoelectric buzzer.

3. The method of claim 2, wherein the timer controller controls a voltage applied to the piezoelectric buzzer.

4. The method of claim 1, wherein the binary pulse data controls at least one of a frequency and a volume of the audio output.

5. The method of claim 1, wherein the audio device is included in a wireless device worn a patient.

6. The method of claim 1, wherein the audio output comprises an auditory notification, and wherein each type of auditory notification corresponds to a different audio file.

7. The method of claim 1, further comprising:
configuring, by a capture and compare register, the duty cycle of the output signal.

8. The method of claim 7, wherein pulse width modulation (PWM) is used to configure the duty cycle of the output signal.

9. An audio device comprising:
a non-volatile storage device that stores binary pulse data, wherein the binary pulse data is generated by converting an audio file;
a central processing unit that retrieves the binary pulse data from the non-volatile storage device and populates a buffer in memory;
a direct memory access (DMA) engine that transfers the binary pulse data stored in the non-volatile storage device to at least one electronic component; and
the at least one electronic component that controls a duty cycle of an output signal of the audio device based on the binary pulse data to emit an audio output.

10. The audio device of claim 9, wherein the audio output is outputted using a piezoelectric buzzer.

11. The audio device of claim 9, wherein the at least one electronic component controls a voltage applied to the piezoelectric buzzer.

12. The audio device of claim 9, wherein the binary pulse data controls at least one of a frequency and a volume of the audio output.

13. The audio device of claim 9, wherein the audio device is included in a wireless device worn by a patient.

14. The audio device of claim 9, wherein the audio output comprises an auditory notification, and wherein each type of auditory notification corresponds to a different audio file.

15. The audio device of claim 9, further comprising:
a capture and compare register that configures the duty cycle of the output signal.

16. The audio device of claim 15, wherein pulse width modulation (PWM) is used to configure the duty cycle of the output signal.

17. A wireless device comprising:
an audio device to:
convert an audio file into binary pulse data, wherein the binary pulse data is stored in a non-volatile storage;
retrieve, via a processor, the binary pulse data from the non-volatile storage and populate a buffer in memory;
transfer, via a direct memory access (DMA) engine, the binary pulse data to a timer controller of the audio device; and
control a duty cycle of an output signal of the audio device based on the binary pulse data to emit an audio output.

18. The wireless device of claim 17, wherein the audio device includes a piezoelectric buzzer.

19. The wireless device of claim 17, wherein a capture and compare register uses pulse width modulation (PWM) to configure the duty cycle of the output signal to control a voltage applied to the piezoelectric buzzer to control at least one of a frequency and a volume of the audio output.

20. The wireless device of claim 17, wherein the audio output comprises an auditory notification, and wherein each type of auditory notification corresponds to a different audio file.
